(19) United States
(12) Patent Application Publication
(10) Pub. No.: US 2025/0032058 A1
(43) Pub. Date: Jan. 30, 2025

(54) METHOD FOR PERFORMING SCANS USING A COMPUTED TOMOGRAPHY SYSTEM

(71) Applicant: Siemens Healthineers AG, Forchheim (DE)

(72) Inventors: Fahri SIMSEKLI, Forchheim (DE); Hannes BRUCHNER, Fuerth (DE); Claus KOEGLER, Hemhofen (DE)

(73) Assignee: Siemens Healthineers AG, Forchheim (DE)

(21) Appl. No.: 18/783,494

(22) Filed: Jul. 25, 2024

(30) Foreign Application Priority Data
Jul. 28, 2023 (DE) 10 2023 207 224.6

(51) Int. Cl. A61B 6/03; A61B 6/00

(52) U.S. Cl. CPC A61B 6/03; A61B 6/54

(57) ABSTRACT

One or more example embodiments relates to a method for performing scans via a computed tomography system, wherein the method comprises rotating a rotor of a gantry of the computed tomography system relative to a patient; performing a topogram scan via the computed tomography system including recording projection data which enables an overview of at least a part of the patient; establishing settings for parameters for a main scan based on the projection data, the parameters for the main scan specifying at least one of at least one irradiation power level or an examination region for the main scan; and performing the main scan of the patient using the computed tomography system based on the parameters for the main scan, wherein the topogram scan and the main scan are performed during the rotating.

### CROSS-REFERENCE TO RELATED APPLICATION(S)

The present application claims priority under 35 U.S.C. § 119 to German Patent Application No. 10 2023 207 224.6, filed Jul. 28, 2023, the entire contents of which is incorporated herein by reference.

### FIELD

One or more example embodiments relates to a method for performing scans via a computed tomography system. One or more example embodiments also relates to a control facility for controlling a computed tomography system and a computed tomography system.

### RELATED ART

In a CT (computed tomography) examination, before the main scan, a topogram is often performed in order to be able to define parameters for the main scan. These parameters are, for example, the posture of the patient, his height and body volume and are identified as “SSDE information”. This serves for the protection of the patient since the dose that a patient is to expect during the main scan must be at least roughly estimated or a plan for the main scan must exist, so that the patient is not scanned wrongly, for example, with too low a dose or in a false position. In principle, the parameters that must be used in the main scan in order to perform it successfully and optimally for the patient and to achieve usable results must be determined.

This takes place in the context of a separate topogram scan and conventionally is performed with a static rotor of the gantry of the CT system.

For the topogram scan, the X-ray source and the collimator is set to 0°, 90°, 180° or 270°, switched on and then the patient support is displaced with the rotor static in order to scan a region of the patient or even the whole patient. The topogram scan is performed with a constant voltage and a constant current for the X-ray source, for example, at a power output of the X-ray source of approximately 5.5 kW at a voltage of 100 kV and a current of 55 mA or 7 kW (50 mA at 140 kV). A single topogram scan, for example at the angle 0°, can be performed, two topogram scans at different angles or more topogram scans, wherein care must be taken that each topogram scan requires a particular time and represents a particular radiation burden for the patient.

For a 500 mm-long topogram, at a table advance speed of 20 cm with a setting of the angle and production of the recordings, approximately 3 s to 5 s per topogram scan is required. Therein, approximately 2000 to 3000 recordings are produced per second, each representing a subregion (corresponding to the width of the detector) of, for example, 10 mm to 60 mm of the sampled region and overlapping to a large extent. Faulty recordings can then be eliminated by averaging.

Once the topogram has been created and the parameters for the main scan are known, the rotor of the gantry must be accelerated to a particular rotation velocity for the main scan. Once the rotor has reached its rotation target velocity (e.g. 2 to 4 rotations per second), the main scan, e.g. a spiral scan, can be performed. The acceleration procedure between the topogram scan and the main scan can last as long as 30 s to 40 s. This means that a pause of 30 s to 40 s occurs between the topogram scan and the main scan. During this time, the patient must lie still on the table and must not move.

Following the main scan (or a plurality of main scans for this patient), the rotor of the gantry must be decelerated again for the next topogram scan and brought to a standstill. A deceleration procedure typically also lasts between 30 s and 40 s. Following deceleration, the CT system must be brought into the target position for the next topogram scan.

### SUMMARY

This means that the rotor of the gantry is decelerated after every main scan and thereafter must be accelerated again for the next main scan. In this way, there arise not only waiting times which, particularly where a CT system is heavily utilized, are disadvantageous, but also a mechanical wear on the gantry from the accelerating forces (in particular the decelerations) and an increased current consumption for the acceleration procedures.

One or more example embodiments provide an alternative to the conventional performance of CT scans. This is achieved via a method according to claim 1, a control facility according to claim 10, and a CT system according to claim 13. Independent of the grammatical term usage, individuals with male, female or other gender identities are included within the term.

### DETAILED DESCRIPTION

Example embodiments will be described again in greater detail, making reference to the accompanying drawings. In the various drawings, the same components are provided with identical reference signs. The drawings are, in general, not to scale. In the drawings:

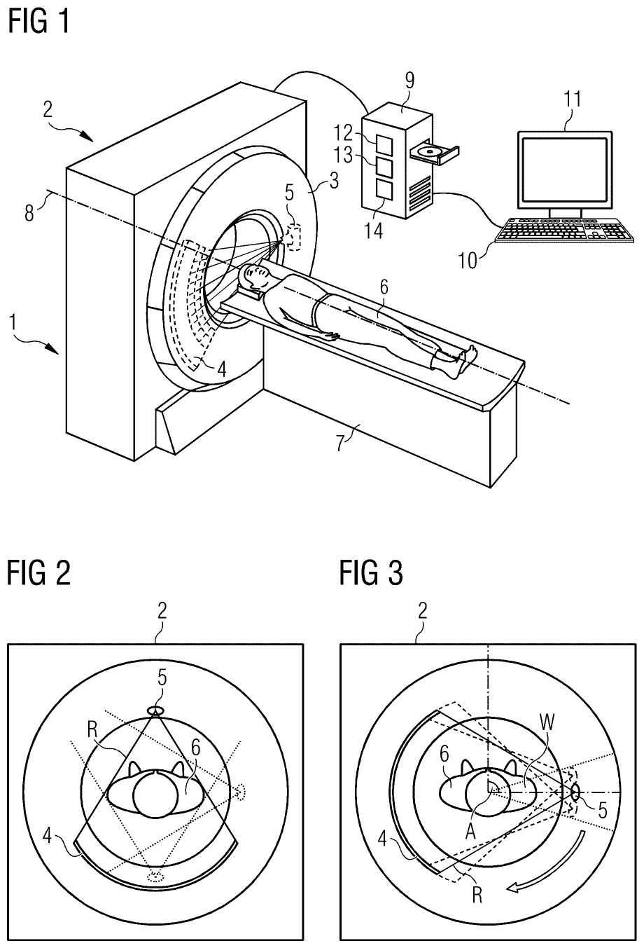

FIG. 1 shows a rough schematic representation of a CT system with an exemplary embodiment of a control facility for performing the method,

FIG. 2 shows possible positions for a topogram scan,

FIG. 3 shows a topogram scan,

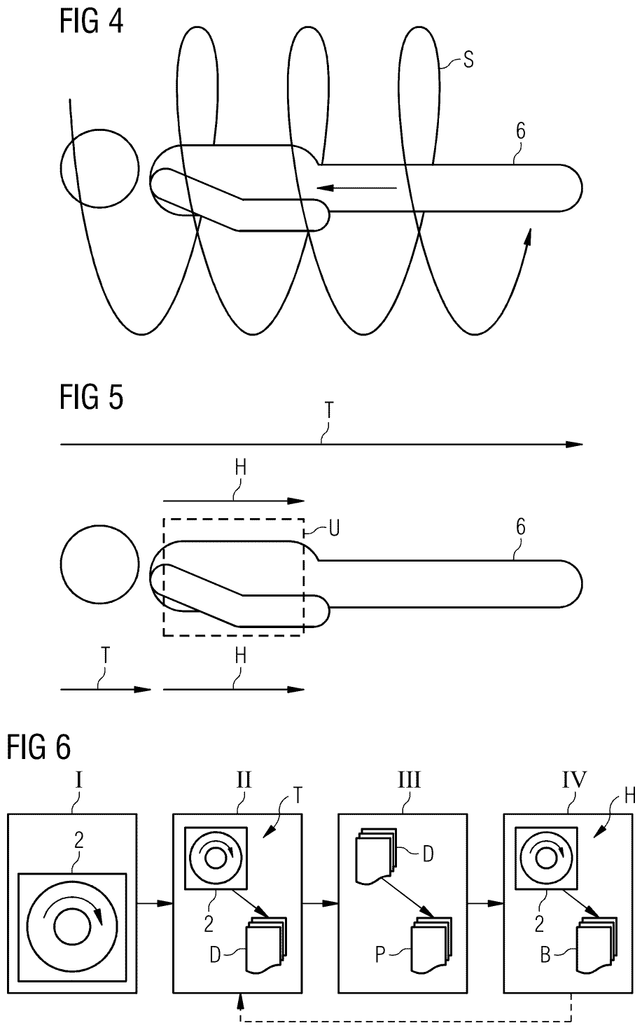

FIG. 4 shows a sketch of a spiral scan,

FIG. 5 shows two possibilities for using the topogram scan for a main scan, and

FIG. 6 shows a block flow diagram for a preferred exemplary embodiment of a method according to the invention.

### DETAILED DESCRIPTION

One or more example embodiments relates to a method for performing scans via a computed tomography system, wherein the method comprises the following steps:
bringing a rotor of a gantry of the computed tomography system into a rotation relative to a patient,
performing a topogram scan via the computed tomography system, wherein projection data which enables an overview of at least a part of the patient is recorded,
establishing settings for parameters for a main scan on the basis of the projection data, wherein the parameters specify at least one irradiation power level and/or an examination region for the main scan,
performing the main scan of the patient via the computed tomography system on the basis of the parameters, in particular, on the basis of the settings for the parameters, wherein both the topogram scan and also the main scan are performed during the rotation of the rotor.

In particular, it can be provided that the parameters for the main scan are set on the basis of the settings established for the parameters for the main scan. In particular, it can be provided that the gantry of the computed tomography system has the rotor, a support structure, a rotary bearing and a rotary drive, that the rotor is mounted via the rotary bearing to be rotatable relative to the support structure about a rotation axis and that the rotary drive is configured for driving the rotation of the rotor about the rotation axis.

In particular, it can be provided that the rotor has an X-ray source for generating X-ray radiation and a detector for detecting the X-ray radiation and/or that the projection data is recorded on the basis of the X-ray radiation. It can further be provided that when the main scan of the patient is performed via the computed tomography system, computed tomography imaging data is recorded, in particular, on the basis of the X-ray radiation.

The principles for controlling a computed tomography system are known in the prior art, in particular, the performance of a scan and the data recording performed therein. It is also known what radiation power levels and accelerating voltages must be used for a particular type of scan. It is known, for example, which radiation parameters a topogram scan should have for recording projection data which enables an overview of at least one part of a patient. Further embodiments are outlined below, but this topogram scan can be performed with a conventionally used X-ray radiation power level.

The establishment of settings for parameters for a main scan from the projection data of the topogram scan is also known, in principle, to a person skilled in the art and is currently performed in examinations. These parameters are typically an irradiation power level and/or an examination region for the main scan, although it is always stated which region of the patient is to be investigated (e.g. lung or heart), but it is not necessarily known exactly where this examination region is situated in the patient and whether the patient is positioned correctly. The examination region must therefore be adapted, for example, to the position in the patient of the organ to be investigated and the position of the patient on the patient support (his isocenter).

The performance of the main scan of the patient with the computed tomography system is also known in principle in the prior art. The peculiarity of one or more example embodiments is that both the topogram scan and also the main scan are performed with the rotor of the gantry of the computed tomography system rotating. Thereby, both a waiting time for an acceleration or deceleration between the scans is avoided and the wear is reduced.

Often, spiral scans are performed in which the gantry and the patient support are displaced relative to one another, in particular at a constant velocity while the rotor of the gantry is rotating. Herein, the rotation of the rotor of the gantry should be harmonized with the feed rate of the patient support and the size of the detector so that a continuous image is generated. For example, during a rotation of the rotor of the gantry at 4 rotations per second, a feed rate of not more than approximately 20 cm/s should be selected so that with a detector having a width of 6 cm, there is sufficient overlap between the individual recordings at the same angular position, so that the images can be assembled well into an overall image. This applies both for the topogram scan and also for the main scan. However, this is not an absolute necessity for the topogram scan, as is described in greater detail below. With a smaller detector, the table velocity can be adapted to the rotation of the rotor of the gantry or the parameters can be established from non-contiguous strip images.

As an alternative to a spiral scan, the patient can be accommodated on a stationary patient support and the patient support can be displaced relative to the gantry between the scans.

A topogram scan should thus be performed during the rotation of the rotor of the gantry. The radiator therein preferably releases the radiation only at pre-defined radiator/collimator positions (at the recording angles). If the user selects a 90° position, the radiator starts to radiate at, for example 85° and ends the radiation at 95°. The patient support should move the same distance as the detector width at each rotation. An image reconstruction unit preferably evaluates the values at the prevailing generator current. If these values rise above a particular value (e.g. “full dose” and/or “parameterized angle”), it is assumed that recording is undertaken with a continuous X-ray radiation and the recorded data should then be reconstructed by the image reconstruction unit as CT images.

Account should be taken of the fact that with conventional topogram scans, the full detector width is often not used, but only a thin strip (due to the overlapping of the recordings). With the method described here, it is preferred that for the topogram scan the full detector width, that is, the whole detector is used for the recordings.

One or more example embodiments further relates to a control facility for controlling a computed tomography system on the basis of the method according to one or more example embodiments which has an establishing unit, wherein the establishing unit is configured to establish settings for parameters for the main scan on the basis of the projection data, wherein the parameters specify at least the irradiation power output and/or the examination region for the main scan. The control facility is configured to control the bringing of the rotor of the gantry of the computed tomography system into rotation relative to the patient, the performance of the topogram scan via the computed tomography system, wherein the projection data which enable the overview over the at least one part of the patient is recorded, and the performance of the main scan of the patient via the computed tomography system on the basis of the parameters, wherein both the topogram scan and also the main scan are performed during the rotation of the rotor.

A computed tomography system according to one or more example embodiments comprises a control facility according to one or more example embodiments and/or is configured for performing a method according to one or more example embodiments.

Components of the control facility according to one or more example embodiments can be realized entirely or partially in the form of software modules in a processor of a suitable computing system, in particular a computer. A realization largely through software has the advantage that conventionally used computing systems can also easily be upgraded by way of a software update in order to operate in the manner according to one or more example embodiments. In this respect, the object is also achieved via a corresponding computer program product with a computer program which is loadable directly into a computing system, having program portions in order to perform the steps of the method according to one or more example embodiments, at least the steps that can be executed by a computer when the program is executed in the computing system. It should be noted in this regard that the execution of a scan corresponds to the output of corresponding control data and the recording of projection data corresponds to the receiving of the corresponding detector signals. Such a computer program product can comprise, where relevant, apart from the computer program, additional constituents, such as, for example, documentation and/or additional components including hardware components, for example, hardware keys (dongles, etc.) in order to use the software.

For transport to the computing system or to the control facility and/or for storage on or in the computing system or the control facility, a computer-readable medium, for example, a memory stick, a hard disk or another transportable or fixedly installed data carrier can be used on which the program portions of the computer program which can be read in and executed by a computing system are stored. For this purpose, the computing system can have, for example, one or more cooperating microprocessors or suchlike.

It should be noted that method steps such as the performance of a scan can correspond to the issuing of corresponding control commands. Further particularly advantageous embodiments and developments of one or more example embodiments are disclosed by the dependent claims and the following description, wherein the claims of one claim category can also be further developed similarly to the claims and description passages relating to another claim category and, in particular also, individual features of different exemplary embodiments or variants can be combined to new exemplary embodiments or variants.

According to a preferred method, the topogram scan can be performed in a previously specified angle range about a number of pre-determined recording angles of the rotor. The recording angles are preferably the conventionally used angles at the zenith above the patient, at the nadir beneath the patient or from the respective side. The recording angles of the rotor can be, in particular, recording angles of the rotor relative to the support structure about the rotation axis.

In particular, a first recording angle of the number of recording angles can be an angle at which the recording takes place from the zenith above the patient or from the nadir beneath the patient. In particular, a second recording angle of the number of recording angles can lie at 90° or 180° thereto, wherein preferably at the second recording angle, fewer recordings are made and/or recordings are made with a lower X-ray radiation power level than at the first recording angle.

It could also be said that the angles are 0°, 90°, 180° or 270°, wherein theoretically it is possible to proceed both from the coordinate system of the gantry and also from the coordinate system of the patient, since the patient can be recorded lying on his back or on his side or on his abdomen. The angle range W specifies the range about this recording angle. It is the case therein that the recording angle lies in the center of the angle range, so that at a recording angle A, there is a recording angle range from A−W/2 to A+W/2. At a preferred angle range of 10° about the recording angle ranges 0° and 90°, the topogram scan would be performed in the angle ranges 355° to 5° and 85° to 95°.

It is preferable therein that one of the recording angles is an angle from which the recording takes place at the zenith above the patient or at the nadir beneath the patient. Dependent upon the type of examination, however, a recording angle of 90° or 270° can also be advantageous. A combination of 0°/180° with a recording at 90° or 270° can also be preferable. In this regard, it should be considered how important, for example, a correct positioning of the patient is, as against an additional radiation burden from an additional topogram scan. Often, the recording angle is also dependent upon the type of the recording. For example, it is preferred to record the lung from above or beneath, but the head from the side.

In the following, the expression “recording angle” should also always be understood to include “recording angle range”. Therein, the recording angle is the center of the recording angle range. Recordings at a recording angle take place in the recording angle range.

If recordings are made from different recording angles, it is then particularly preferred that in a first recording angle, recordings for the topogram are prepared and at the other recording angle, fewer recordings are made and/or recordings are made at a lower X-ray radiation power level. For example, the topogram scan is performed from a recording angle of 0° (in an angle range of) 10° and a few recordings (at a lower radiation dose, where required) are made from a recording angle rotated by 90° thereto in order to determine selectively the correct position of the isocenter. For this purpose, it should be noted that a position at the start of the main scan and parameters relating to the position of organs can be derived from a topogram scan. However, the isocenter or additional information regarding the volume of the patient can be established merely from shadows cast by the body.

According to a preferred variant of the preceding embodiment, the topogram scan is performed only at one recording angle and in combination therewith, a camera recording is performed from a recording angle at 90° rotated thereto in the visible spectrum or the infrared spectrum which reproduces at least the contours of the patient. So that the camera is not exposed to the radiation, the camera position can be displaced along the rotation axis of the rotor of the gantry relative to the X-ray source, that is, in front of or behind the X-ray source. Even if patients are sometimes surrounded by covers or mats during a recording, a camera is particularly advantageous in order to be able to perform, for example, a rough estimate of the patient size and the organ position. For example, the head is usually readily recognizable on camera images. The topogram can also correct the camera images.

But the camera images can also complement a topogram. For this purpose, it should be noted that a topogram should not take more than 10 s. In the case of very narrow detectors, for example, with a thickness of 1 cm, at a fixed rotation rate of the rotor (e.g. 2 revolutions per s), this time could be exceeded or merely “strip recordings” of the patient could be made in which regions between the strips are not shown in the topogram. With the camera images, it is then possible to extrapolate a complete topogram from the strip recordings, in particular using a model. In a preferred case in which additionally a camera (mounted stationary above, beneath or beside the patient) records the patient during the topogram scan, with a rapid movement of the table, at least a type of outer contour of the patient can be reproduced and inner structures would then be present in strip form or complemented from the model. The result would suffice, however, at least to estimate the isocenter and the volume of the patient and also to specify the spatial beginning of the main scan. Thus, the additional use of a camera represents a variant in which even with low-cost devices (with small detectors and/or low rotation velocities of the rotor), the method can be performed without difficulty.

It is therefore preferred that, simultaneously with the topogram scan, the patient is recorded by a camera and the camera recordings are united with the topogram scan. The possibilities described above, in particular, can be regarded as “unification”. It should be noted that a checking of the isocenter must not necessarily take place via recording angles rotated by 90°, rather a size comparison can be made from recordings the recording angles of which are 180° apart. The recordings should overlap so that the comparisons can be performed with the same body region.

According to a preferred method, an angle range of less than 20° (e.g. with a recording angle of 0°, the range would then be from 350° to) 10°, preferably less than 10°, in particular less than 5° or even less than 2° (that is, for example, from 359° to 1°). Even if the angle range for each recording angle can be different, it is preferred that for recordings from a plurality of recording angles, the angle ranges about all the recording angles are of equal size.

Preferably, a plurality of recordings are made within an angle range, preferably more than 2 recordings, but preferably more than 50 recordings. This has the advantage that in the event that a recording is faulty, it can be replaced by another recording or the recordings can be averaged to reduce errors. For example, at a recording angle of 0°, three recordings can be made, one at 359°, one at 0° and one at 1° and the angle range would thus be 2° here. Finally, the number of recordings can be determined from the error probability of a CT system.

According to a preferred method, at rotation angles of the rotor outside the respective angle ranges about the number of the recording angles, an X-ray source of the computed tomography system used for recording is switched off. This has the great advantage that the radiation burden on the patient is reduced. Therein, the same acceleration voltage and the same current can certainly be used for the X-ray source as for a conventional topogram scan. However, for a conventional topogram scan, the X-ray source is constantly switched on. In the embodiment described here, only “flash recordings” are being made in the relevant angle ranges and the X-ray source is otherwise switched off if no recordings are made (outside the angle ranges). It is therein preferred that the X-ray source is switched on only for the making of a number of recordings in the respective angle ranges. If, therefore, recordings are made in an angle range N, then in this case, the X-ray source should be switched off between the recordings. Here, however, the time that the X-ray source needs for stable emission of an X-ray beam should be taken into account. If the angle range is traveled through within this time due to the rotation of the rotor, then it would be advantageous if the X-ray source remained switched off within the angle range. The quality of a topogram recorded in this way corresponds, even with a reduced X-ray radiation power level, to the quality of conventional topograms. The rotation angles of the rotor can be, in particular, rotation angles of the rotor relative to the support structure about the rotation axis.

According to a preferred method, both the topogram scan and also the main scan are each performed in the form of a spiral scan in that via a patient support of the computed tomography system, a translation movement of the patient relative to the gantry of the computed tomography system is performed, in particular, continuously, that is, at a constant velocity, while the rotor rotates.

According to a preferred method, before the execution of the topogram scan, an examination region is defined and it is established, on the basis of the projection data of the topogram scan, whether the examination region has been reached. This can take place according to the overall topogram scan, although it is particularly advantageous if it takes place during the topogram scan. For this purpose, for example, each image can be searched through for a particular structure. For example, it is known before the topogram scan which region of the patient is to be investigated. Now, each individual image of the topogram can be investigated automatically after its recording and it can be ascertained which part of the patient this image shows. Since the fundamental anatomy of a patient does not differ from other patients, it can be estimated when the examination region could have been reached or at least whether the examination region has been reached. In practice, this can take place, for example, via an automated image recognition and a comparison with a model of the anatomy of a patient.

On reaching the examination region, the X-ray radiation power level for the topogram scan is preferably increased. In this regard, it should be considered that, on the basis of images which show only rough features of the patient, for example the outline, it is possible to estimate whether the examination region has been reached. Therefore, the topogram scan could initially be performed with a reduced power level and, if the examination region has been reached, at a (previously defined) normal power level. The reduced power level is preferably less than 80% of the normal power level, in particular less than 60% or even less than 50%. In particular, in the case of a spiral scan as the topogram scan, this procedure is advantageous, in particular, since then the examination can be performed better.

However, it is also possible to start the main scan on reaching the examination region. Herein, it should be noted that the main scan is performed with a continuous beam. If the beam is temporarily switched off during the topogram scan, it should then be operated continuously for the main scan.

According to a preferred method, before the timepoint of the topogram scan reaching the examination region, the settings for the parameters for the main scan are established using a model of the patient and the projection data recorded in the topogram scan; and the performing the main scan is started based on the parameters for the main scan upon the timepoint of the topogram scan reaching the examination region. The parameters can be established, in particular, via a model of the patient, wherein the model can originate from a prior examination and/or can be calculated by modifying a basic model via the projection data of the topogram scan.

For example, in the case of an examination of the lung with a spiral scan, it can be established via the topogram scan whether the bronchi have been reached. Until the bronchi are reached, from the already scanned portion of the patient (head and shoulders), the size and position of the patient can be estimated. It can be established therefrom whether the patient lies correctly and which pre-settings for radiation parameters are to be used for the main scan. If it is discernible that the topogram scan has reached the examination region, in particular by recognizing characteristic structures or by evaluation from the previously scanned structures and the feed rate of the patient support, the main scan can be started and the corresponding X-ray radiation power level can be adjusted and recordings can be made at many different angles.

Preferably, individual images of the main scan are used as (projection) images of a continued topogram scan. These are the recordings that have been made at the angle ranges about the recording angles at which the original topogram scan also took place. On the basis of these images, it is then decided, in particular, when the main scan will be ended. Via this continued topogram scan, it can be established when the examination region has been passed through. It should be noted in this regard that from topograms (that is, projection images), information can be calculated more quickly than from tomograms (reconstructed 3D images). With smaller detectors, for example, with a strip width of 1 cm, representations of the just recorded part of topograms is possible almost in real time. The performing the main scan based on the parameters may be started while continuing the topogram scan after the topogram scan reaching the examination region, i. e., without terminating or interrupting the topogram scan when reaching the examination region.

It should be noted that the patient support can certainly be halted and moved backwards during a scan. Thus, if it has been discovered during a topogram scan that the limits of the examination region have already been exceeded, the patient support could be moved back a little way, in particular, with the X-ray source switched off and then the main scan could be started at the correct position.

According to a preferred method, a model of a patient or a subregion of a patient is selected on the basis of the projection data of the topogram scan and/or is created by way of a modification of a basic model. A selection can take place from among a large number of patient models which have been created in advance. The most similar model is then selected. However, a basic model can be provided and this can then be adapted to the patient by way of geometric changes, in particular scalings, rotations, stretchings or compressions. A combination is also conceivable which is selected from a plurality of models which comes closest to the physique of the patient and this is then used as a basic model for a modification.

It is preferred therein that the model is created during the topogram scan (and thus not thereafter) on the basis of the previously recorded projection data and this created model is modified with further projection data recorded in the context of the topogram scan.

A preferred control facility comprises a model unit, wherein the model unit is configured for
selecting a model of a patient or a subregion of a patient (possibly as a basic model) and/or for
modifying a basic model
   on the basis of the projection data.

As far as a patient model is concerned, a simple basic model can be used in which body regions are formed by basic geometrical bodies such as ellipsoids or spheres, general cylinders or cylinders or possibly also cubes. The size of the individual basic bodies can be established by adapting the model to the topogram. Herein, however, not necessarily all parts of the model are to be adapted. For example, the length of the legs does not play a major role for a lung scan, although the shape and the volume of the upper body does. It is often possible, using partial recordings of the body of a patient to adapt the entire model, at least in a manner that is essential for the main scan (and the establishing of pre-settings for the parameters thereof).

According to a preferred method, a plurality of examinations, each comprising a topogram scan and a main scan are performed successively. Therein, the rotor preferably rotates at a constant velocity from the first to the last examination. Therein, a plurality of patients is preferably investigated. The peculiarity is that the rotor rotates throughout the whole time, for example, from the beginning of the examinations in the morning until the end of the examinations in the evening.

It is therein preferred that during a pause between two examinations, the rotor rotates in an idling mode in which the rotation is maintained and/or in which energy is expended neither for an acceleration nor for a deceleration. It could be discussed, in particular at this point, when the next examination should take place and an energy could be estimated which would have to be expended for an acceleration if the rotor were to rotate in idling mode and how much energy a maintenance of the rotation would require. An idling rotation or a maintenance could depend upon the result of this estimation.

A preferred control facility comprises an idling unit configured to maintain the rotation of the rotor of the gantry between two examinations substantially without an energy feed and without initiating a deceleration. The expression “substantially” means that no more than enough energy should be used such that the rotation velocity of the rotor remains constant and, in particular, no energy is expended for an acceleration.

An advantage of one or more example embodiments is that the rotor no longer has to be decelerated and accelerated between scans. This means a time saving, less wear and a lower current consumption. A continuous rotation of the CT system is possible. After a topogram scan, the main scan can be started immediately without delays. A patient must lie in the recording position for a shorter time. In addition, a significant reduction in the radiation can be obtained if the X-ray source switches off during the recordings of the topogram scan. Despite a lower radiation emission, the same or a better image quality of the topogram scan can be achieved.

FIG. 1 shows an embodiment of a computed tomography (CT) system 1 with a detector 4 and an X-ray source 5. The X-ray source 5 is configured to irradiate the detector 4 with X-ray radiation. The CT system 1 shown comprises a gantry 2 with a rotor 3. The rotor 3 has the X-ray source 5 and the detector 4. The detector 4 is configured to detect the X-ray radiation.

The rotor 3 is rotatable about the rotation axis 8 which herein can simultaneously be regarded as the patient longitudinal axis. The patient 6 is positioned on the patient support 7 and is movable along the rotation axis 8 through the gantry 2. The control facility 9 is provided for controlling the computed tomography system 1.

In a main scan, typically, computed tomography imaging data from the object 6 is recorded via the detector 4 from a large number of angular directions at one radiation energy. Subsequently, on the basis of the computed tomography imaging data, via a mathematical method, for example, comprising a filtered back projection or an iterative reconstruction method, a final X-ray image dataset (“CT images”) can be reconstructed.

The control facility 9 can further have an image reconstruction unit for reconstructing an image dataset on the basis of the computed tomography imaging data. In addition, an input facility 10 and an output facility 11 are connected to the control facility 9. The input facility 10 and the output facility 11 can, for example, enable an interaction by way of a user or the representation of a generated image dataset or can output a problem solution that has been established.

The control facility 9 herein comprises an establishing unit 12 which is configured for establishing settings for parameters P for a main scan H from the projection data D of a topogram scan T, wherein the parameters P specify at least one irradiation power level and/or an examination region U for the main scan H.

The control facility 9 is configured to perform a topogram scan T and a main scan H, with a rotating rotor 2. The topogram scan T serves to record projection data D, which enables an overview of at least part of a patient 6. The main scan H serves to record computed tomography imaging data which can be reconstructed, for example, to an image dataset.

The control facility 9 also comprises a model unit 13 which is configured for selecting a basic model of a patient 6 and for modifying this basic model. Both the selection and also the modification therein take place on the basis of projection data D of the topogram scan T.

In addition, the control facility 9 herein comprises an idling unit 14 which is configured to maintain the rotation of the rotor 3 of the gantry 2 between two examinations substantially without an energy feed and without initiating a deceleration.

FIG. 2 shows possible positions for a topogram scan T. What is indicated is a recording from above wherein the X-ray source 5 of the gantry 2 is arranged directly above the patient 6 and the detector 4 is directly thereunder. This position can be regarded, for example, as 0°. The X-ray radiation R, in the form of a cone, is therein intended to irradiate the whole patient 6 in a strip which corresponds to the detector area. Shown dotted are two further possible positions in which the X-ray source 3 is positioned beside the patient 6 (90°) or beneath the patient 6 (180°).

FIG. 3 shows a sketch of a topogram scan T. Herein, the rotor 3 rotates in the direction of the arrow and the recordings are here made from the side at 90°. The recording angle A of 90° relative to a vertical line is indicated (dot-dashed line). The recordings of the projection data D take place within an angle range W about the recording angle A (dotted lines). A position of the X-ray source 3 and the detector 4 at the recording angle A is shown and, with dashed lines, two further possible positions within the angle range W for two further recordings.

FIG. 4 shows a sketch of a spiral scan in which the X-ray source 3 rotates together with the detector 4 as part of the rotor 3 (see e.g. FIG. 1) about the patient 6, while the patient is moved along the arrow on a displaceable patient support 7 through the gantry 2. From the point of view of the patient, the X-ray source then describes a spiral path S. The movement of the patient can take place, for example, at 20 cm/s while the rotor 3 rotates at four rotations per second.

FIG. 5 shows two possibilities for using the topogram scan T for a main scan H. At top, it is indicated with arrows that firstly the topogram scan T extends over the whole body of the patient 6 and then the main scan H is performed over the examination region U. At bottom, it is indicated with arrows that firstly the topogram scan T extends over a part of the patient 6 and when the examination region U is reached, the main scan H is performed. The topogram scan T can therein be continued with projection images of the main scan H which have been recorded at the recording angles A of the topogram scan T.

FIG. 6 shows a block flow diagram for a preferred exemplary embodiment of a method according to one or more example embodiments for controlling a computed tomography system 1 (see FIG. 1).

In step I, a rotor is brought into rotation so that the subsequent steps can be performed with a rotating rotor.

In step II, a topogram scan T is performed to record projection data D with the rotor 3 rotating. This projection data D is intended to enable an overview of at least a part of a patient 6.

The topogram scan T is performed in a previously specified angle range W about a number of pre-determined recording angles A of the rotor 3 (see FIG. 3). At each angle range, a plurality of recordings is made, for example, three to ten. By way of example, the topogram scan T takes place at the angle 0° and a few recordings are made with a reduced dose from 90° in order to determine the isocenter of the patient 6.

In order to reduce the dose to the patient 6, the X-ray source 5 of the gantry 2 used for recording is switched off at rotation angles of the rotor 3 outside the respective angle range W about the recording angle A.

Now, on the basis of the projection data D of the topogram scan T, a basic model for the patient 6 can be selected and this basic model can be modified such that it is adapted to the patient 6. It can thereby be established where the examination region U (see, for example, FIG. 5) in the patient 6 should lie and what radiation dose can and should be administered.

In step III, such settings for parameters P for a main scan H are established from the projection data D of the topogram scan T. These parameters P herein specify an irradiation power level and an examination region U for the main scan H.

In this step, in particular on the basis of the projection data D of the topogram scan T, it can be determined whether the examination region U has been reached and, if relevant, as shown in FIG. 5 below, in a positive case, the main scan H is started.

In step IV, the main scan H of the patient 6 is then performed via the computed tomography system 1, wherein the computed tomography imaging data B are recorded.

The steps II to IV can be performed multiple times in succession for a plurality of patients 6, as the dashed arrow below is intended to indicate. Meanwhile, the rotor 3 rotates evenly without being decelerated and accelerated again between the examinations.

Finally, it should again be noted that the drawings described above in detail merely involve exemplary embodiments which can be modified by a person skilled in the art in a wide variety of ways without departing from the scope of the invention. Furthermore, the use of the indefinite article “a” or “an” does not preclude the possibility that the relevant features can also be present plurally. Similarly, the expressions “unit” and “facility” do not preclude the components in question from consisting of a plurality of cooperating partial components which can also be spatially distributed. The expression “a number” is to be understood as meaning “at least one”.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program or code, instructions, some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for performing scans using a computed tomography system, the method comprising:
rotating a rotor of a gantry of the computed tomography system relative to a patient;
performing a topogram scan via the computed tomography system including recording projection data which enables an overview of at least a part of the patient;
establishing settings for parameters for a main scan based on the projection data, the parameters for the main scan specifying at least one of at least one irradiation power level or an examination region for the main scan; and
performing the main scan of the patient using the computed tomography system based on the parameters for the main scan, wherein the topogram scan and the main scan are performed during the rotating.

2. The method of claim 1, wherein the performing the topogram scan performs the topogram scan in a previously specified angle range about a number of pre-determined recording angles of the rotor.

3. The method of claim 2, wherein the angle range is less than 20°.

4. The method of claim 2, wherein at rotation angles of the rotor outside the respective angle ranges about the number of the recording angles, an X-ray source of the computed tomography system used for recording is switched off.

5. The method of claim 1, wherein the performing the topogram scan and the performing the main scan are each performed in a spiral scan, the method further comprising:
performing a translation movement of the patient relative to the gantry of the computed tomography system during the rotating.

6. The method of claim 1, wherein an examination region is defined before the performing the topogram scan, the method further comprising:
determining whether the examination region has been reached based on the projection data of the topogram scan; and
increasing an X-ray radiation power level for the topogram scan on reaching the examination region.

7. The method of claim 6, further comprising:
before the topogram scan reaching the examination region, establishing the settings for the parameters for the main scan using a model of the patient and the projection data recorded in the topogram scan; and
starting the performing the main scan based on the parameters for the main scan upon the topogram scan reaching the examination region.

8. The method of claim 1, further comprising:
at least one of selecting a model of the patient or a subregion of the patient based on the projection data of the topogram scan or creating the model of the patient or the subregion of the patient by way of a modification of a basic model.

9. The method of claim 1, wherein a plurality of examinations, each comprising a topogram scan and a main scan, are performed in succession and the gantry is rotated at a constant velocity from a first examination to a last examination.

10. A control facility for controlling a computed tomography system to perform the method of claim 1, the control facility comprising:
an establishing unit configured to establish settings for the parameters for the main scan based on the projection data, wherein the parameters for the main scan specify at least one of at least the irradiation power level or the examination region for the main scan, the control facility being further configured to,
control the rotation of the rotor,
control the performing of the topogram scan, and
control the performing of the main scan of the patient.

11. The control facility of claim 10, further comprising:
a model unit, wherein the model unit is configured to at least one of
select a model of the patient or a subregion of the patient based on the projection data, or
modify a basic model based on the projection data.

12. The control facility of claim 10, further comprising:
an idling unit, wherein the idling unit is configured to maintain the rotation of the rotor of the gantry between two examinations substantially without an energy feed and without initiating a deceleration.

13. A computed tomography system, comprising the control facility of claim 10.

14. A non-transitory computer program product comprising commands which, when executed by a computer, cause said computer to cause the computed tomography system to perform the method of claim 1.

15. A non-transitory computer-readable storage medium comprising commands which, when executed by a computer, cause said computer to cause the computed tomography system to perform the method of claim 1.

16. The method of claim 3, wherein at rotation angles of the rotor outside the respective angle ranges about the number of the recording angles, an X-ray source of the computed tomography system used for recording is switched off.

17. The method of claim 16, wherein the performing the topogram scan and the performing the main scan are each performed in a spiral scan, the method further comprising:
performing a translation movement of the patient relative to the gantry of the computed tomography system during the rotating.

18. The method of claim 17, wherein an examination region is defined before the performing the topogram scan, the method further comprising:
determining whether the examination region has been reached based on the projection data of the topogram scan; and
increasing an X-ray radiation power level for the topogram scan on reaching the examination region.
